(19) United States
(12) Patent Application Publication
(10) Pub. No.: US 2014/0200463 A1
(43) Pub. Date: Jul. 17, 2014

(54) MENTAL STATE WELL BEING MONITORING

(71) Applicant: Affectiva, Inc., Waltham, MA (US)

(72) Inventors: Rana el Kaliouby, Boston, MA (US); Daniel Abraham Bender, Cambridge, MA (US)

(73) Assignee: Affectiva, Inc., Waltham, MA (US)

(21) Appl. No.: 14/214,751

(22) Filed: Mar. 15, 2014

Related U.S. Application Data
(63) Continuation-in-part of application No. 13/153,745, filed on Jun. 6, 2011.
(60) Provisional application No. 61/798,731, filed on Mar. 15, 2013.
(60) Provisional application No. 61/793,761, filed on Mar. 15, 2013.
(60) Provisional application No. 61/789,038, filed on Mar. 15, 2013.
(60) Provisional application No. 61/790,461, filed on Mar. 15, 2013.
(60) Provisional application No. 61/844,478, filed on Jul. 10, 2013.
(60) Provisional application No. 61/916,190, filed on Dec. 14, 2013.
(60) Provisional application No. 61/924,252, filed on Jan. 7, 2014.
(60) Provisional application No. 61/927,481, filed on Jan. 15, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/16; A61B 5/0205; A61B 5/01; A61B 5/00; A61B 5/11

(52) U.S. Cl. CPC A61B 5/165; A61B 5/0022; A61B 5/486; A61B 5/1103; A61B 5/01; A61B 5/02055; A61B 5/6898; A61B 5/7278
USPC 600/484; 600/300; 600/595; 600/508; 600/558; 600/547; 600/549; 600/529; 600/483

(57) ABSTRACT

The mental state of an individual is obtained to determine their well-being status. The mental state is derived from an analysis of facial information and physiological information of an individual. The well-being status of other individuals is correlated to the well-being status of the first individual. The well-being status of the individual or group of individuals is rendered for display. The well-being status of an individual is used to provide feedback and to recommend activities for the individual.

### RELATED APPLICATIONS

This application claims the benefit of U.S. provisional patent applications “Mental State Well Being Monitoring” Ser. No. 61/798,731, filed Mar. 15, 2013, “Mental State Analysis Using Heart Rate Collection Based on Video Imagery” Ser. No. 61/793,761, filed Mar. 15, 2013, “Mental State Analysis Using Blink Rate” Ser. No. 61/789,038, filed Mar. 15, 2013, “Mental State Data Tagging for Data Collected from Multiple Sources” Ser. No. 61/790,461, filed Mar. 15, 2013, “Personal Emotional Profile Generation” Ser. No. 61/844,478, filed Jul. 10, 2013, “Heart Rate Variability Evaluation for Mental State Analysis” Ser. No. 61/916,190, filed Dec. 14, 2013, “Mental State Analysis Using an Application Programming Interface” Ser. No. 61/924,252, filed Jan. 7, 2014, and “Mental State Analysis for Norm Generation” Ser. No. 61/927,481, filed Jan. 15, 2014. This application is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. The foregoing applications are each hereby incorporated by reference in their entirety.

### FIELD OF ART

This application relates generally to the analysis of mental states and more particularly to the monitoring of mental state well-being.

### BACKGROUND

An individual's mental state is important to general well-being and effective decision making However, the mental state of an individual might not always be apparent to that individual. Mental states include a wide range of emotions and experiences from happiness to sadness, from contentedness to worry, from excitation to calm, and many others. As these mental states are often experienced in response to everyday events, changes in an individual's mental state often are not easily recognizable. Though an individual can often perceive his or her own emotional state quickly, instinctively and with a minimum of conscious effort, the individual might encounter difficulty when attempting to summarize or communicate his or her mental state to others. Individuals are often aware of their mental state based on interactions with others and general observations. The ability and means by which one person perceives his or her emotional state can be quite difficult to summarize. Knowledge and identification of a person's mental state can allow for the re-evaluation of certain decisions, the changing of certain activities, or even the cessation of specific activities.

Many mental states such as frustration, confusion, disappointment, boredom, disgust, and delight can be identified to aid in understanding the outlook of an individual or group of individuals. For example, individuals can respond individually and collectively with fear and anxiety when presented with certain disturbing stimuli, such as a catastrophic event. Similarly, people can respond with happy enthusiasm to an event they perceive positively, such as their favorite sports team winning an important victory. When an individual is aware of his or her mental well-being, he or she is better equipped to realize his or her own abilities, cope with the normal stresses of life, work productively and fruitfully, and contribute to his or her community.

### SUMMARY

A computer can be used to collect mental state data from an individual, analyze the mental state data, and render an output that provides the well-being status of the individual. The well-being status may then be presented to the individual as feedback which may include recommending activities, eliminating activities, and identifying a potentially impaired state. A computer-implemented method for mental state analysis is disclosed comprising: obtaining mental state data on an individual; analyzing the mental state data to evaluate a well-being status for the individual; and rendering an output based on the well-being status.

The rendering may include posting the well-being status to a social network. The method may further comprise querying for well-being statuses across the social network. The well-being status may provide input to a recommendation engine. The method may further comprise aggregating the well-being status for the individual with well-being statuses for a plurality of other people. The method may further comprise correlating the well-being statuses with activities performed by the plurality of other people.

In embodiments, a computer program product embodied in a non-transitory computer readable medium for mental state analysis comprises: code for obtaining mental state data on an individual; code for analyzing the mental state data to evaluate a well-being status for the individual; and code for rendering an output based on the well-being status. In some embodiments, a computer system for mental state analysis comprises: a memory which stores instructions; one or more processors attached to the memory wherein the one or more processors, when executing the instructions which are stored, are configured to: obtain mental state data on an individual; analyze the mental state data to evaluate a well-being status for the individual; and render an output based on the well-being status. In embodiments, a computer-implemented method for mental state analysis comprises: receiving mental state data on an individual; analyzing the mental state data to evaluate a well-being status for the individual; and sending the well-being status for rendering. In some embodiments, a computer-implemented method for mental state analysis may comprise: capturing mental state data on an individual; analyzing the mental state data to provide mental state information; and sending the mental state information to a server for analyzing wherein the analyzing will provide a well-being status for the individual and wherein the well-being status will be rendered. In embodiments, a computer-implemented method for mental state analysis comprises: receiving a well-being status based on mental state data obtained on an individual wherein the well-being status results from analyzing the mental state data to provide the well-being status for the individual; and rendering an output based on the well-being status.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for mental state well-being monitoring.

FIG. 2 is a flow diagram for well-being status usage.

FIG. 3 is an example social network page with feedback.

FIG. 4 is an example emotional profile screen.

FIG. 5 is an example dashboard with well-being status shown.

FIG. 6 is an example response interaction to well-being status.

FIG. 7 is an example showing collection of mental state data.

FIG. 8 is an example biosensor on a person.

FIG. 9 is a system diagram for mental state well-being monitoring.

### DETAILED DESCRIPTION

People exhibit and communicate a wide range of mental states. These mental states include various emotions and emotional responses, and are often encountered in reaction to everyday events. Changes in the mental state of an individual can occur quickly and can be difficult to recognize. Because of this difficulty, an individual might struggle to summarize and describe their mental state. Providing an individual with an assessment of their well-being can assist the individual with decision-making, activity selection, activity scheduling, and other recommended activities. If and when such an assessment describes an impaired state, individuals can make informed choices to eliminate certain activities which could be causing the impaired state. Well-being can be a state including being happy, successful, healthy, content, or in a generally positive mood. A well-being status can be an indication of whether someone has a generally positive attitude or conversely generally negative. The well-being status can be a reflection of valence or emotion.

Analysis of an individual's mental state can provide a means by which an individual can view feedback regarding the status of their well-being. The analysis can take place using a computer with which the user is interacting; the computer(s) that captured the sensor data; and/or from one or more other computers, which can be local or remote to the user. This feedback can include recommendations for different activities, and can include recommendations for activity performance based upon time of day, a period of time during the day, or another type of calendar-based scheduling. The well-being status can also be included in an aggregated analysis with a plurality of people that can result in recommendations for activities.

Current systems for analyzing the mental state of many individuals do not scale well to the evaluation of a single person. For example, one traditional measurement method for quantifying valence levels—valence representing an indication of whether a person is positively or negatively disposed—requires individuals on a panel to turn a hardware dial to quantify valence throughout a television show. In such studies, dial values, which can be collected at discrete time intervals such as once per second, can range from 0 to 100. An individual might be told that a value of 0 indicates disinterest, a value of 50 indicates a neutral mental state, and a value of 100 indicates interest in the television show. Such a system has many drawbacks, as its reliability is based upon aggregated continuous measurement from large consumer panels. Also, the data is one-dimensional; there is no way to collect data about various and concurrently experienced mental states including excitement, happiness, surprise and disappointment, all of which can correspond to positive valance.

Well-being analysis can be performed by evaluating facial expressions, hand gestures, and physiological conditions exhibited by an individual. The human face is a powerful channel for communicating a wide variety of emotional states. The general expressiveness of an individual as they view input stimuli can be analyzed to determine a well-being state. A camera or another facial recognition device can be used to capture images of an individual's face, and software can be used to extract and interpret laughs, smiles, frowns, and other facial expressions.

Other physiological data can also be useful in determining the mental state well-being of an individual. Gestures, eye movement, perspiration, electrodermal activity (EDA), heart rate, blood pressure, and respiration are a few examples. A variety of sensor types can be used to capture physiological data, including heart rate monitors, blood pressure monitors, EDA sensors, or other types of sensors. A camera can be useful for capturing physiological data and facial images simultaneously. Sensors coupled to a computer—in some embodiments, the same computer with which the user is interacting; in other embodiments, one or more other computers—are able to detect, capture, and/or measure one or more external manifestations of a user's mental state. For example, in certain embodiments a still camera is able to capture images of the user's face; a video camera is able to capture images of the user's movements; a heart rate monitor is able to measure the user's heart rate; a skin-resistance sensor is able to detect changes in the user's electrodermal activity; and an accelerometer is able to measure such movements as gestures, foot tapping, or head tilts, to name a few. In embodiments, multiple sensors to capture the user's mental state data can be included.

Once the data has been collected from the individual, an analysis of the mental state data is obtained. The analysis can take place on the computer with which the user is interacting, on the computer(s) that captured the sensor data, and/or on one or more other computers which can be local or remote to the user. The analysis can provide the mental states of the user over time. In some cases, the mental state of the user can be estimated. Mental state information, based on physiological data, can be used to augment or replace data captured from a camera. In some embodiments, self-report methods of capturing mental state information, such as the previously mentioned dial approach, are also used in conjunction with the mental state information captured from cameras, sensors, monitors, or other equipment.

Once the mental state well-being information has been produced, an output can be rendered to the individual. The rendering can include data and analysis, both of which can be posted on a social-network web page. The data and/or analysis can describe the well-being status of the individual. The data and/or analysis can also describe recommendations for the individual. The recommendations can include activities such as watching a video, playing a game, or participating in a social activity, to name a few. The results of the mental state analysis can also be included in a calendar where the results can be displayed or compared with the ongoing activities already included in the calendar. The analysis can comprise correlating the well-being status of an individual to a particular activity. The analysis can include aggregating the well-being status of the individual with the well-being statuses of a plurality of other people, and can further correlate the well-being statuses of a plurality of people with activities performed by the plurality of people.

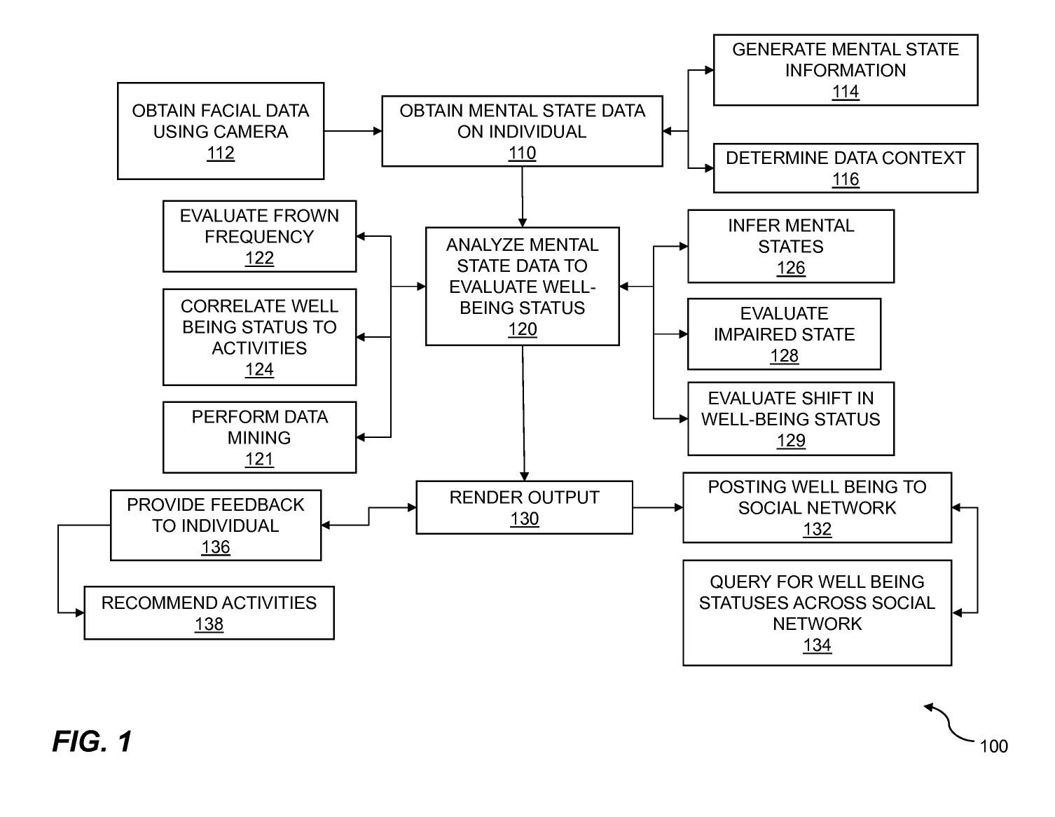

FIG. 1 is a flow diagram for mental state well-being monitoring. A flow 100, which describes a computer-implemented method for mental state analysis, is shown. The flow 100 includes obtaining mental state data on an individual 110. The collecting of mental state data can be performed by numerous methods in various embodiments, and can include capturing facial images of an individual as they respond to stimuli. Facial image data can be obtained using a camera 112, but other types of image-capture devices can be used as a source of facial data, including a webcam, a video camera, a still camera, a thermal imager, a CCD device, a phone camera, a three-dimensional camera, a depth camera, multiple webcams used to show different views of a person, or any other type of image capture apparatus that can allow captured data to be used in an electronic system. In some embodiments, the data is collected from multiple sources. The mental state data may include facial data, physiological data, accelerometer data, or the like. The mental state data can comprise various types of data including, but not limited to, heart rate, respiration rate, blood pressure, skin conductance, audible sounds, gestures, or any other type of data that can be useful for determining mental state information. Thus, in some embodiments, the mental state data includes electrodermal activity data. In embodiments, the mental state data is obtained as a background task by a device. A device can be performing other tasks, such as facilitating a cell phone call, while the mental state data is collected and stored or communicated to another computing device.

In some embodiments, the mental state information is generated 114 in the form of a description or summary. The flow 100 can further comprise determining contextual information 116 related to the collected mental state data. Various types of contextual information can be obtained including time of day, activity, other people in proximity, current news events, and the like. Some examples of contextual information that can be collected include a task assigned to the user; the location of the user; the environmental conditions to which the user is exposed, such as temperature, humidity, and the like; the name of the content being viewed; the level of noise experienced by the user; or any other type of contextual information. The location can be determined via GPS or other location sensing. In some embodiments, the contextual information is based on one or more of skin temperature and accelerometer data. In some embodiments, the contextual information is based on one or more of a photograph, an email, a text message, a phone log, or GPS information. In embodiments, information on the context can be tagged to the mental state data for future reference.

The flow 100 includes analyzing the mental state data 120 to evaluate a well-being status for the individual. The analyzing of the mental state data to evaluate the well-being status can be accomplished in an ongoing fashion. The analyzing of mental state data 120 can include various type of analysis, including computation of means, modes, standard deviations or other statistical calculations over time. The analyzing of mental state data 120 can include inferring mental states 126, which can be a type of mental state information. The mental state can be factored into a well-being status evaluation. The mental state data can include one or more of smiles, laughter, smirks or grimaces. Mental state data can be collected sporadically or continually over a time period. In embodiments, mental state data is analyzed to infer mental states such as frustration, confusion, disappointment, hesitation, cognitive overload, focusing, engagement, attention, boredom, exploration, confidence, trust, delight, disgust, skepticism, doubt, satisfaction, excitement, laughter, calmness, sadness, happiness, stress, anger, fatigue, and curiosity.

An embodiment of mental state data analysis includes an evaluation of certain facial expression frequency 122 and other facial data. The analyzing can include evaluating frown frequency, smile frequency, laugh frequency, or the like. Analysis can be as simple as tracking when someone smiles or when someone frowns. Facial data can include data on a subject tilting his or her head to the side, leaning forward, smiling, a frowning, as well as many other gestures or expressions. Tilting the head forward can indicate that a viewer is engaged with a media presentation or another form of stimuli. A furrowed brow can indicate concentration. A smile can indicate being positively disposed or a state of happiness. Laughing can indicate that an individual is experiencing enjoyment and that an individual finds a particular subject humorous. A tilt of the head to the side and a furrow of the brows can indicate confusion. A horizontal shake of the head can indicate displeasure. Mental states such as these and many others can be inferred using collected mental state data on an individual, including captured facial expressions and physiological data. In some embodiments, physiological data, accelerometer readings, and facial data are each used as contributing factors in algorithms that infer various mental states.

The flow can further comprise a correlation of well-being status to activities 124. The data which was captured can be correlated to an activity the individual is performing. The activity can be one comprising an interaction with a web site, a movie, a movie trailer, a product, a computer game, a video game, a personal game console, a cell phone, a mobile device, an advertisement, or another action, such as consuming food. Interaction can refer to passive viewing or active viewing and responding. The well-being status of an individual can be used in emotional journaling. An individual might find that writing his or her emotions down on paper can help them to process difficult times as well as help them to sort out general emotional problems. The well-being status of an individual can be used as input to a recommendation engine. A recommendation engine can provide relevant, individual recommendations based upon the well-being status of an individual or plurality of individuals. The recommendation engine can take advantage of contextual information or other relevant data. The recommendation engine may suggest playing a certain game in response to a certain well-being status indication. The recommendation engine may modify a game based on the well-being status indication. In embodiments, the recommendation engine suggests articles or other material to read based on the well-being status indication. In embodiments, the recommendation engine suggests various media to view or interact with based on the well-being status indication. The recommendation engine may suggest music or other audio presentation. In some embodiments, the music or other audio is turned on at an appropriate volume level by the recommendation engine. The recommendation engine may suggest taking a jog or performing some other exercise routine. The recommendation engine may share some media with others based on the well-being status. For instance, a significant positive shift in well-being status when a certain movie or other media is being viewed could enable the recommendation engine to share information on that media to others. That shared information could be provided to others in your social network or could be anonymously or collected in aggregation to obscure an individual's identity.

In some embodiments, the flow 100 includes the evaluation of an impaired state 128. The impaired state can be a function of fatigue, illegal drugs, over-the-counter drugs, prescription drugs, alcohol, distraction, and the like. An impaired mental state can include significant impairment of intelligence and social functioning. In some cases, the impaired state can be associated with abnormally aggressive or irresponsible conduct. In some embodiments, an analysis that includes a determination of an impaired state leads to recommendations being made for the individual.

The flow 100 can include evaluating a shift in well-being status 129. Identifying a change in well-being status as well as the context during the change can be helpful in recommending future activities to participate in or to avoid. The shift may be correlated to a certain event, such as an improvement in well-being in response to a visit by a family member or friend. The shift may be compared to previous events such as performing an exercise routine. The shift may be compared to previous shifts in well-being status. By mining these previous shifts an activity may be identified as problematic, such as a negative shift in well-being status after a redeye flight. The shift can be in comparison to a recent well-being status or can be in comparison to the same time of day, the same day of the week, the same season of year, or in comparison to some other relevant period of time. As indicated, the flow can include performing data mining 121 on previous mental state data and evaluating contributing factors toward the well-being status.

The flow 100 includes rendering an output 130 displaying the well-being status. In various embodiments, the rendering is graphical, pictorial, textual, auditory, or any combination thereof. The rendering can be presented on a local display or a remote electronic display. In some embodiments the rendering is printed on paper. The flow 100 can further comprise posting information based on the analysis to a social network page 132. The flow 100 can further comprise querying for well-being statuses across the social network 134. The flow 100 can also comprise querying in light of a context. The rendering can include an aggregated analysis with a plurality of people. The feedback can include aggregating the well-being status of the individual with the well-being statuses of a plurality of other people, and, in embodiments, correlating the well-being statuses of a plurality of people with activities performed by this plurality. The process can include analyzing information from the plurality of other individuals wherein the information allows for the evaluation of the mental state of each of the plurality of other individuals and the correlation of the mental state of each of the plurality of other individuals to the mental state data which was captured on the individual.

The rendering can include providing feedback to the individual 136. In embodiments, the feedback describes the well-being status of the individual. The mental state can be presented on a webpage, computer display, electronic display, cell phone display, the screen of a personal digital assistant (PDA), or another display. The mental state can be displayed graphically. A series of mental states can be presented along with the likelihood of each state for a given point in time. Likewise a series of probabilities for each mental state can be presented over the timeline for which facial and physiological data was analyzed.

The feedback can describe recommended activities 138. In some embodiments, an action can be recommended based on the mental state which was detected. The recommended activities can include one or more of watching a video, playing a game, or participating in a social function. The activities can be recommended for a period of time or a time of day. The activity can be included in a calendar query that can be displayed or compared with the ongoing activities already included in the calendar.

The feedback can recommend eliminating an activity. In some embodiments the feedback recommends that an action be terminated or eliminated. The feedback can also indicate that the activity should be eliminated for a period of time or time of day. The feedback to eliminate an activity can be included in a calendar query and can be displayed or compared with the ongoing activities already included in the calendar. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

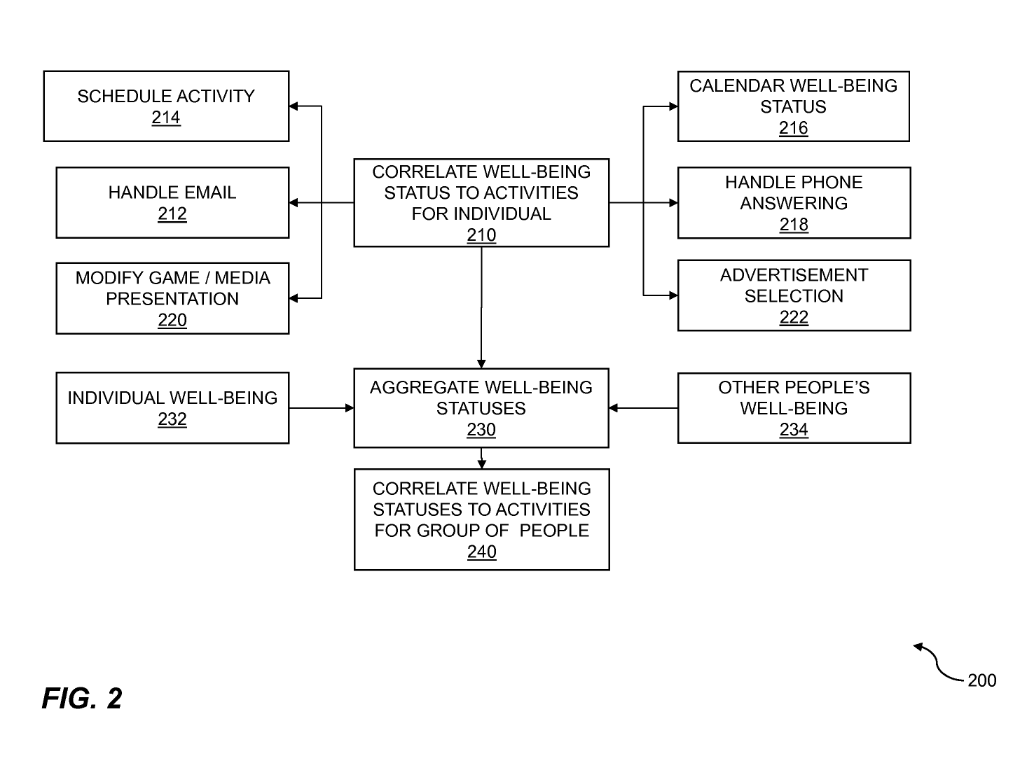

FIG. 2 is a flow diagram for well-being status usage. A flow 200 can continue from or be part of the previous flow 100. A wide variety of uses for well-being statuses can be considered, only some of which are shown in FIG. 2. The flow 200 can include correlating the well-being status to activities performed by the individual 210. The activities can include one or more of watching a video; playing a game; participating in a social function; interacting with a website, a movie, a product, a computer game, a videogame, a cell phone, a mobile device, or an advertisement; or an activity such as eating. The flow 200 can further include calendaring the well-being status 216. The well-being status can relate to activity performed based on a time of day, a period of time during the day, or another form of calendar-based scheduling. In some embodiments, the flow 200 includes scheduling an activity 214 on a calendar based on the well-being status. The flow 200 can also include recommending a movie, video, game, social activity, or another activity based on the mental state information correlating to the well-being status of the individual. For example, if the well-being status of an individual indicates stress, then a relaxing activity can be recommended. Similarly, if the well-being status of an individual indicates a state of heightened perception, then a business activity can be scheduled. If an individual demonstrates a positive well-being status when performing a particular activity, a recommendation of a similar activity can be provided.

The flow 200 can further comprise handling phone answering 218 based on the well-being status. In some embodiments, the well-being status is displayed when an individual answers the phone. In other embodiments, the well-being status is displayed at the conclusion of a telephone conversation or is aggregated, analyzed, and displayed after a plurality of telephone conversations over a period of time, such as a day. By identifying the well-being status of a person answering the phone, recommendations can be made to improve the attitude of the phone answerer. In some embodiments, the well-being status of an individual is displayed prior to the moment an individual answers a telephone. The well-being status of an individual can also be used to change phone answering activities when an individual is not on a phone conversation. For example, the voicemail message of an individual can change based upon their well-being status. For certain well-being statuses, the phone system can be programmed to present a “Do Not Disturb” message. In embodiments, certain well-being statuses restrict and filter potential phone callers, in some cases only allowing certain callers such as family or friends to connect. A system using the well-being status to screen callers could ask whether the call was an emergency, and ring through to the individual if that was the case.

The flow 200 can further comprise handling email 212 based on the well-being status. In some embodiments, the well-being status is displayed while an individual is reading or composing an email. In some embodiments, the well-being status of an individual is displayed prior to the moment an individual begins to compose or answer an email. In other embodiments, the well-being status is displayed at the conclusion of an email activity, or aggregated, analyzed, and displayed after a plurality of email activities over a period of time, such as a day. By identifying the well-being status of a person reading or composing an email, recommendations can be made to improve the attitude of the individual. The well-being status can also be used to modify activities, other than sending and receiving messages, pertaining to dealing with email. For example, emails can be filtered or prioritized based upon the well-being status of an individual. If the well-being status of an individual indicates a state of heightened perception, emails requiring thought or concentration can be filtered and prioritized. Another example of filtering email based upon the well-being status of an individual could prioritize junk mail, which can be quickly deleted, in order to provide the individual with a sense of accomplishment.

The well-being status 200 can further comprise advertisement selection 222. An advertisement can be shown to an individual because the individual had a positive well-being state in response to certain similar advertisements. Conversely, an advertisement can be shown to an individual because the individual responded to advertisements of a different type with a negative well-being mental state, thus prompting an attempt to evoke a more positive state by presenting a different style of advertisement. In some embodiments, an advertisement that correlates to the well-being state of an individual based upon a period of time, time of day, or other calendar time frame is made to an individual. Advertisement timing can be chosen based upon the well-being status of an individual. For example, by picking the correct time point for an advertisement based upon the well-being status of an individual, viewers can be retained through commercial breaks in a program. Various types of mental state information can be used to automatically determine advertisement placement, such as excitement, interest, or other well-being state information. In other embodiments, the advertisements can be offered in different locations, with well-being mental state data collected in order to determine which advertisement placement generated the most desirable well-being status.

The well-being status can be used to modify a game 220. The game can be a digital game, a computer game, a video game, a computer game, a game on a personal game machine, an educational game, a multiplayer game, or the like. Modifications to the game can include speeding up the game to cause more focus or attention on the game, slowing down the game to reduce frustration, changing a difficulty level, changing a color scheme to be livelier, changing music to be mellower, or numerous other types of changes. Modifying a game 220 based upon the well-being status of an individual or plurality of individuals can take many forms. The modifying of a game can include changing the tasks with which the individual is presented. The changing of tasks can include making the game harder or easier. The modifying of the game can include changing a role for the individual. For example, when a person starts to exhibit mental states associated with tedium, their role can be changed within the game. The well-being status of an individual can be collected while the individual is involved in the game. The collecting of well-being status can comprise the collecting of one or more of facial data, physiological data, and actigraphy data. In some embodiments, the data is collected by a gaming machine which is part of the gaming environment. In other embodiments, the data is collected by a peripheral device or computer which has access to the individual. In some embodiments, a web camera is used to capture one or more of the facial data and the physiological data. In some embodiments, the physiological data and actigraphy data are obtained from one or more biosensors attached to an individual.

The flow can further comprise analyzing the mental state data of an individual during a game to produce a well-being status. The analysis can be performed by a computer that is remote from the game machine. The analyzing can include aggregating well-being status information with the well-being statuses of others who are playing or have played the game. The mental state information can include valence and arousal. Some analysis can be performed on the client computer before the data is uploaded to a web server, while some analysis can be performed on a server computer. Analysis of the mental state data can take many forms, and can be based on one person or a plurality of people. Communication of the well-being status of an individual can occur in real-time while the game is being played. In some embodiments, the game is modified based on this real-time well-being status communication. Alternatively, communication of the well-being status of an individual or plurality of individuals can occur after the game is completed or after a specific session or goal of the game is completed.

The well-being status can be used to modify a media presentation 220. The media can include any type of content including broadcast media, digital media, electronic media, multimedia, news media, print media, published media, recorded media, social media, and other forms of media content. The well-being status of an individual or plurality of individuals can be determined as they are watching or interacting with the media. In some embodiments, the media presentation is prepared with different versions, and, depending on the goal of the media presentation, mental state data can be collected as the different versions are presented in order to determine which media presentation generates the most positive or negative affect data. Other embodiments use well-being status information to determine the duration for the media presentation. In other embodiments, the well-being status information is used to determine the location of a media presentation. In some embodiments, the media presentation is optimized for a specific platform, such as a mobile phone, tablet computer, or mobile device. Other embodiments optimize the media presentation for a home TV screen, a large movie theater screen, or a personal computer screen, based upon the analysis of an individual's well-being status as they view various media on different devices.

The flow 200 further comprises aggregating the well-being status for an individual 230 with well-being statuses for a plurality of other people. Well-being state data that can be collected includes physiological data, facial data, or any other information gathered about an individual's well-being state. The aggregation can comprise combining various well-being states that were inferred. The aggregation can be a combination of data such as electrodermal activity, heart rate, heart rate variability, respiration, or another type of physiological reading. Aggregating mental state information about the plurality of people can also be performed. An individual can receive aggregated well-being state information from a plurality of people through another computer such as a web-based service. The aggregated information can include the well-being status of an individual 232. The aggregated information can include the well-being status of other people 234.

The flow 200 further comprises correlating the well-being statuses with activities performed by a plurality of other people 240. The activities can include one or more of watching a video; playing a game; participating in a social function; interacting with a website, a movie, a product, a computer game, a videogame, a cell phone, a mobile device, or an advertisement; or consuming food. Information from a larger group of people can be useful in recommending ideas for activities to the individual. The well-being statuses of an individual that can be correlated to an activity 210, as described above, are each also applicable to the correlation of well-being statuses of a plurality of individuals 240. Various steps in the flow 200 may be changed in order, repeated, omitted, or the like without departing from the disclosed concepts. Various embodiments of the flow 200 may be included in a computer program product embodied in a non-transitory computer readable medium that includes code executable by one or more processors.

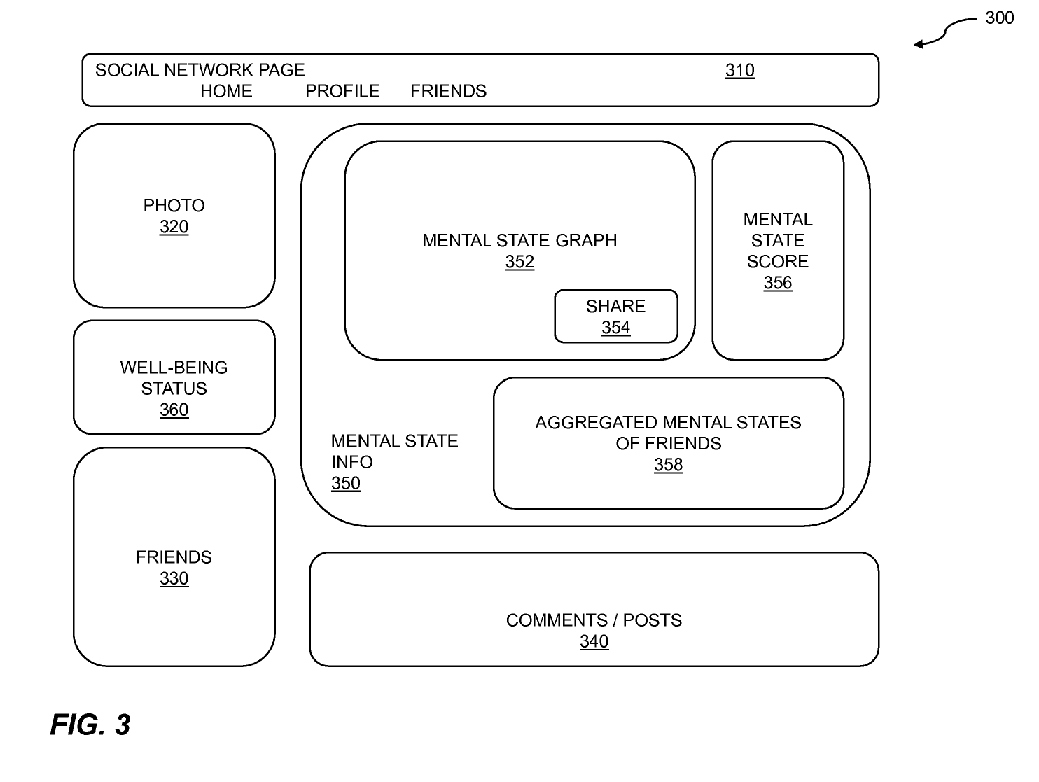

FIG. 3 is an example social network page with feedback. The exact content and formatting can vary between various social networks, but similar content can be formatted for a variety of social networks including, but not limited to, any number of blogging websites, Facebook™, LinkedIn™, MySpace™, Twitter™, Google+™, or any other social network. A social network page for a particular social network can include one or more of the components shown in the example social network page content 300, but can also include various other components in place of, or in addition to, the components shown. The social network content 300 can include a header 310 which can identify the social network and can include various tabs or buttons for navigating the social network site, such as the “Home,” “Profile,” and “Friends” tabs shown. The social network content 300 can also include a profile photo 320 showing the individual who owns the social network content 300. Various embodiments also include a friends list 330 showing the contacts of the individual on the particular social network. Some embodiments include a comments component 340 to show posts from the individual, friends, or other parties.

The social network content 300 can include the well-being status of the individual 360. In various embodiments, the rendering can be graphical, pictorial, textual, auditory, or any combination thereof. In some embodiments, the well-being status is represented by an avatar. The avatar can be selected by the individual, and the avatar can be animated based on the mental state information. For example, if in certain embodiments the individual is excited, the avatar changes to an appearing suggesting excitation.

The social network content 300 can include a mental state information section 350. The mental state information section 350 can allow for posting mental state information to a social-network web page. While in certain embodiments, posted mental state information includes mental state information that has been shared by the individual, other embodiments include mental state information that has been captured but not yet shared. In at least one embodiment, a mental state graph 352 is displayed to the individual showing his or her own mental state information while viewing a web-enabled application. If this mental state information has not yet been shared over the social network, a share button 354 can be included. If the individual clicks on the share button 354, mental state information, such as the mental state graph 352 or various summaries of the mental state information, can be shared over the social network. The mental state information can be shared with an individual, a group or subgroup of contacts or friends, another group defined by the social network, or openly with anyone, depending on the embodiment and the individual's selection. The profile photo 320, or another image shown on the social network, can be updated with an image of the individual demonstrating in some manner the mental state information that is being shared, such as a smiling picture if the mental state information reflects happiness. In some cases, the image of the individual is taken during a peak time of mental state activity. In some embodiments, the photo 320 section, or some other section of the social network page 300, allows for posting video of the individual's reaction or video representing the individual's mental state information along with the photo. If the mental state information shared is related to a web-enabled application, the sharing can include forwarding a reference pertaining to the mental state information to the web-enabled application and can include a URL and a timestamp indicating a specific point in a video. Other embodiments can include an image of material from the web-enabled application or a video of material from the web-enabled application. The forwarding, or sharing, of the various mental state information and related items can be done on a single social network, or some items can be forwarded on one social network while other items can be forwarded on another social network. In some embodiments, the sharing is part of a rating system for the web-enabled application, such as aggregating mental state information from a plurality of users to automatically generate a rating for videos.

Some embodiments include a mental state score 356. In some embodiments, the mental state data is collected over a period of time and the mental state information that is shared is a reflection of a mood for the individual via a mental state score 356. The mental state score can be a number, a sliding scale, a colored scale, various icons or images representing moods, or any other type of representation. Various moods can be represented, including, but not limited to, frustration, confusion, disappointment, hesitation, cognitive overload, focusing, being engaged, attending, boredom, exploration, confidence, trust, delight, and satisfaction.

Some embodiments include a section for aggregated mental states of friends 358. This section can include an aggregated mood of those friends shown in the friends section 330 who have opted to share their mental state information. In some embodiments, the social network page has an interface for querying well-being statuses across the social network. The query can be for people to whom an individual is linked, to friends, to a demographic group, or to some other grouping of people. The embodiments can include aggregated mental states of those friends who have viewed the same web-enabled application as the individual, and some embodiments allow the individual to compare their mental state information in the mental state graph 352 to their friends' aggregated mental state information 358. Other embodiments display various aggregations from different groups.

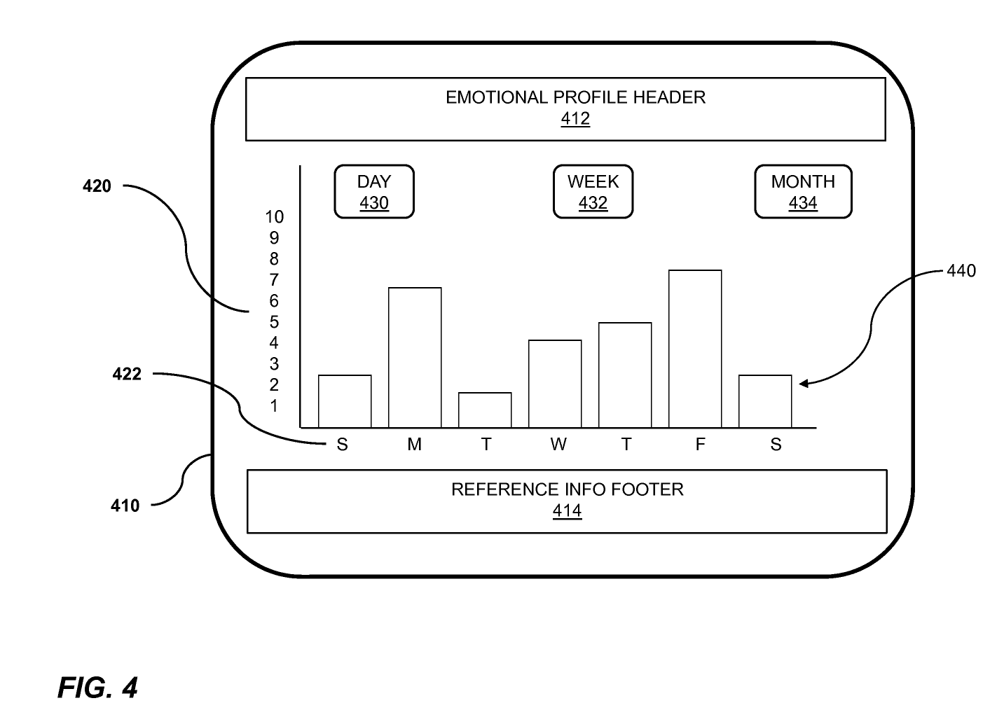

FIG. 4 is an example emotional profile screen. A display, screen, or window 410 is included showing mental state information, well-being status, and emotional profile information. An emotional profile header 412 is included displaying information about the window 410. A reference information footer 414 describes further details about the specific information displayed. Representation for days of the week 422 are shown on the x axis. A scale for representing mental state information is shown on the y axis 420. A button 430 can be selected to show well-being status across a single day. A button 432 can be selected to show well-being status across a week. A button 434 can be selected to show well-being status across a month. In this example, the week button 432 has been selected. A graph 440 is shown describing well-being status for each of the days of the week. Thus, the well-being status may be determined periodically over a period of time and shown on a display. The well-being status can be shown graphically for further analysis or useful representation.

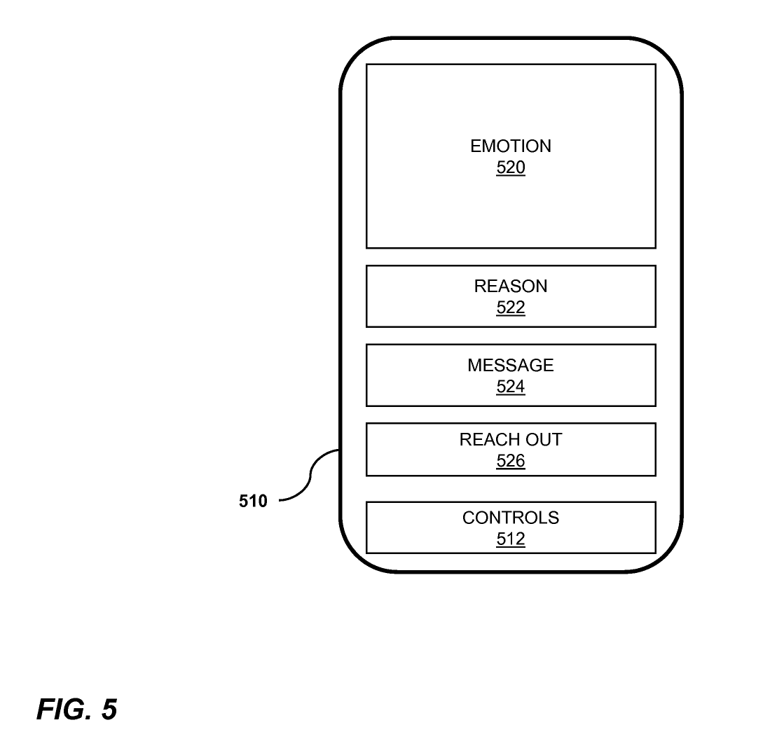

FIG. 5 is an example dashboard with well-being status shown. The dashboard 510 may be a display, screen, or window. In this example, the dashboard 510 is shown as represented on a smartphone device. The dashboard 510 may be shown in response to selecting a certain application or may pop up given predetermined rules or settings on the device or application. An emotion 520 is shown that may represent a well-being status. Some context or reason 522 can be displayed on the dashboard 510. A message 524 from another person, such as a text message, may be displayed on the dashboard 510. For certain well-being status indications, a reach out button 526 may be displayed for possible selection. When such a button is selected, a phone call may be started, a chat session initialized, or the like. For example, if a sadness well-being status is detected and indicated, the cause of the sadness can be displayed, and a reach out can be prompted to a close friend or relative. Various controls 512 can be selected for controlling the handling of well-being status indications and communication.

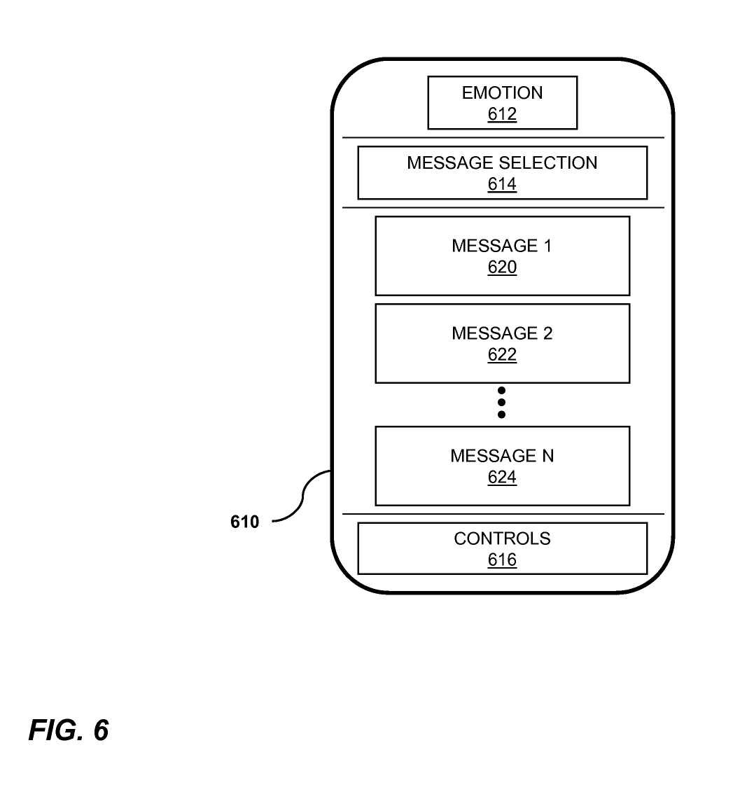

FIG. 6 is an example response interaction to well-being status. A window, screen, or display 610 is shown for offering possible responses to a well-being status indication. An emotion bar 612 is shown that describes a well-being status, such as sadness. A message selection section 614 is shown where possibilities are provided such as recommended articles based on the well-being status. Message 1 620, message 2 622, through message n 624 are shown that describe recommendations. The recommendations can include articles to read, media to view, activities in which to participate, and the like based on the well-being status indication. Various controls 616 can be selected for controlling a response interaction application.

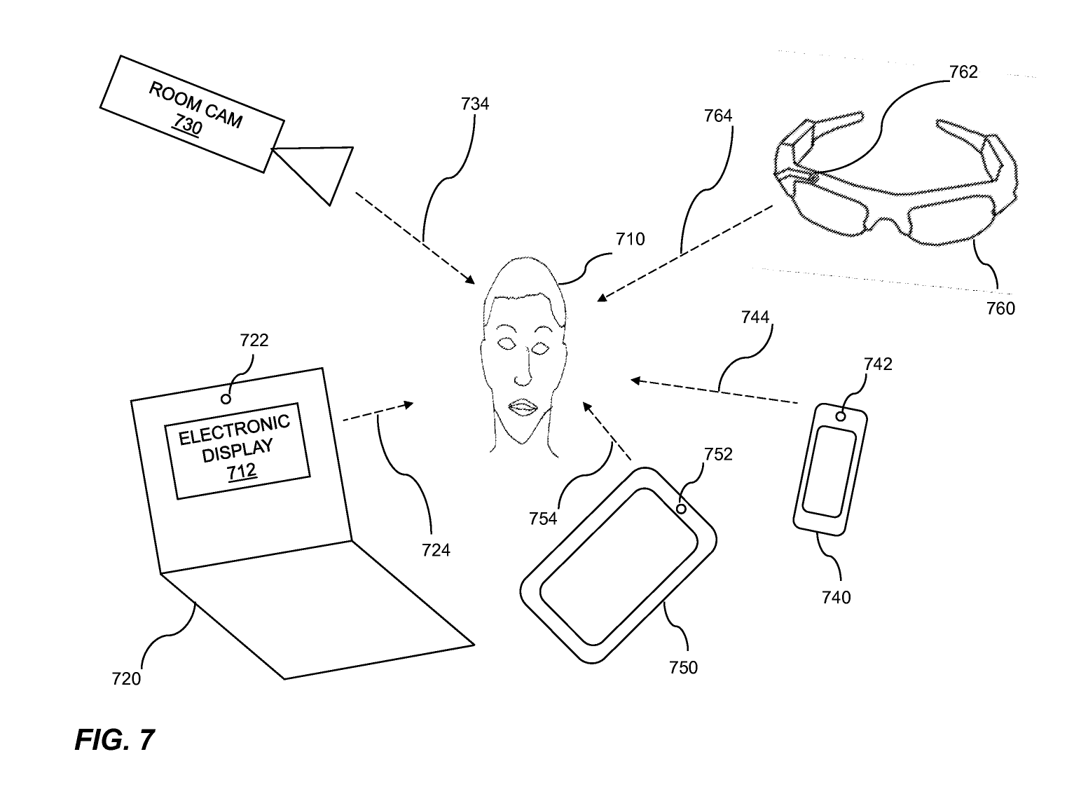

FIG. 7 is an example diagram showing various collection of facial mental state data. A user 710 can be performing a task, such as viewing a media presentation on an electronic display 712, or doing something else where it can be useful to determine the user's mental state. The electronic display 712 can be on a laptop computer 720 as shown, a tablet computer 750, a cell phone 740, a desktop computer monitor, a television, or any other type of electronic device. The display 712 can be any electronic display, including but not limited to, a computer display, a laptop screen, a net-book screen, a tablet computer screen, a cell phone display, a mobile device display, a remote with a display, a television, a projector, or the like. The mental state data can be collected on a mobile device such as a cell phone 740, a tablet computer 750, or a laptop computer 720, or can be collected using a wearable device such as glasses 760. Thus, the multiple sources can include a mobile device, such as a phone 740 or a tablet 750, or a wearable device such as glasses 760. A mobile device can include a forward facing camera and/or a rear facing camera that can be used to collect mental state data. Facial data can be collected from one or more of a webcam 722, a phone camera 742, a tablet camera 752, a wearable camera 762, and a room camera 730.

As the user 710 is monitored, the user 710 might move due to the nature of the task, boredom, distractions, or for another reason. As the user moves, the user's face may be visible from one or more of the multiple sources. Thus if the user 710 is looking in a first direction, the line of sight 724 from the webcam 722 can observe the individual's face, but if the user is looking in a second direction, the line of sight 734 from the room camera 730 can observe the individual's face. Further, if the user is looking in a third direction, the line of sight 744 from the phone camera 742 can observe the individual's face. If the user is looking in a fourth direction, the line of sight 754 from the tablet cam 752 can observe the individual's face. If the user is looking in a fifth direction, the line of sight 764 from the wearable camera 762 can observe the individual's face. A wearable device such as the pair of glasses 760 shown can be worn by another user or an observer. In other embodiments, the wearable device is a device other than glasses, such as an earpiece with a camera, a helmet or hat with a camera, a clip-on camera attached to clothing, or any other type of wearable device with a camera or other sensor for collecting mental state data. The individual 710 can also wear a wearable device including a camera which is used, in embodiments, for gathering contextual information and/or collecting mental state data on other users. Because the individual 710 can move their head, the facial data can be collected intermittently when the individual is looking in a direction of a camera. In some cases, multiple people can be included in the view from one or more cameras, and some embodiments include filtering out faces of one or more other people to determine whether the individual 710 is looking toward a camera.

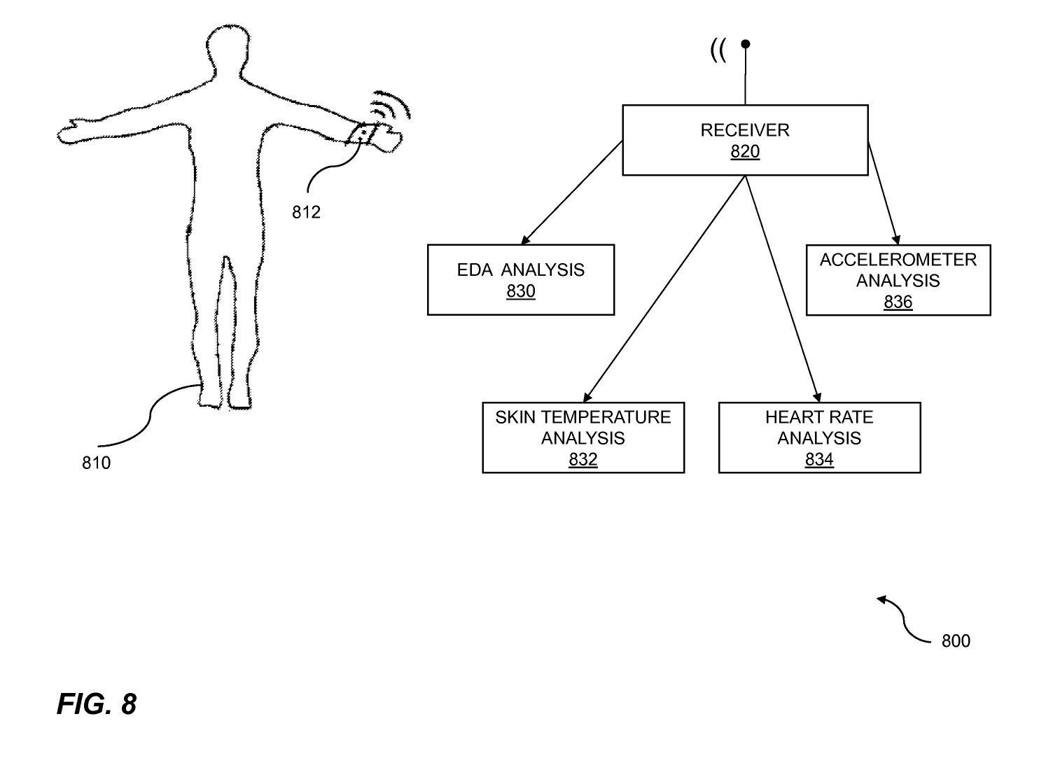

FIG. 8 is an example of a biosensor on a person. A diagram 800 shows various ways a biosensor can provide data about the well-being state of an individual. Physiological data can be gathered from a person 810 to determine their well-being state. In embodiments, a physiological monitoring device 812 is attached to a person 810. The monitoring device 812 can be used to capture a variety of types of physiological data from a person 810 as the person experiences and interacts with various stimuli. The physiological data can include one or more of heart rate, heart rate variability, blink rate, electrodermal activity, skin temperature, respiration, accelerometer data, and the like. The physiological data can be derived from a biosensor. In embodiments, a plurality of people can be monitored as they view and interact with various stimuli.

The person 810 can experience and interact with various stimuli in a variety of ways. Physiological data collected from a person 810 can be transmitted wirelessly to a receiver 820. In embodiments, physiological data from a plurality of people is transmitted to a receiver 820 or to a plurality of receivers. Wireless transmission can be accomplished by any of a variety of means including, but not limited to, IR, Wi-Fi, Bluetooth®, and the like. In embodiments, the physiological data can be sent from a person to a receiver via tethered or wired methods. Various types of analysis can be performed on the physiological data gathered from a person or a plurality of people in order to determine their well-being state. For example, electrodermal activity (EDA) data can be analyzed 830 to identify specific characteristics of an individual's well-being state. The electrodermal activity data can be analyzed to determine a specific activity's peak duration, peak magnitude, onset rate, delay rate, and the like.

Additional types of analysis can be performed on the physiological data gathered from a person or a plurality of people to determine their well-being state. For example, skin-temperature analysis 832 can be performed to measure skin temperature, temperature change rate, temperature trending, and the like. Heart-rate analysis 834 can also be performed. Heart-rate analysis can include heart rate, changes in heart rate, and the like. Further analysis of physiological data can include accelerometer analysis 836. Accelerometer data analysis can include whether or not activities were performed, rate of activity, and the like. In embodiments, other types of analysis are performed on physiological data gathered from a person or a plurality of people to determine the well-being state of an individual or plurality of individuals.

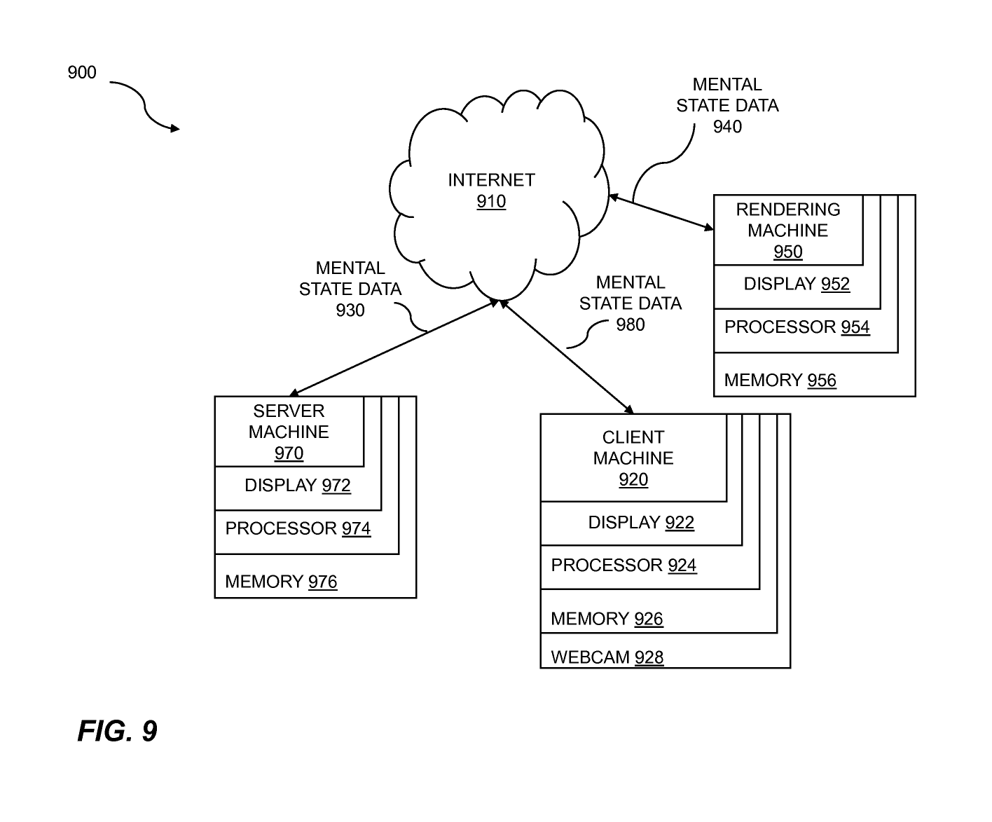

FIG. 9 is a system diagram for mental state well-being monitoring. The diagram illustrates an example system 900 for well-being state collection, analysis, and rendering. The system 900 can include one or more client machines 920 linked to an analysis server 970 via the Internet 910 or another computer network. The example client machine 920 comprises one or more processors 924 coupled to a memory 926 which can store and retrieve instructions, a display 922, and a webcam 928. The memory 926 can be used for storing instructions, mental state data, mental state information, mental state analysis, well-being status indicators, and videos. The display 922 can be any electronic display, including but not limited to, a computer display, a laptop screen, a net-book screen, a tablet computer screen, a cell phone display, a mobile device display, a remote with a display, a television, a projector, or the like. The webcam 928 can comprise a video camera, still camera, thermal imager, CCD device, phone camera, three-dimensional camera, a depth camera, multiple webcams used to show different views of a person, or any other type of image capture apparatus that can allow captured data to be used in an electronic system. The processors 924 of the client machine 920 are, in some embodiments, configured to receive mental state data collected from a plurality of people to analyze the mental state data to produce well-being state information and output the well-being status. In some cases, well-being status can be output real time, based on mental state data captured using the webcam 928. In other embodiments, the processors 924 of the client machine 920 are configured to receive mental state data from one or more people, analyze the mental state data to produce well-being state information, and send a viewer well-being status information including mental state data 980 through the Internet 910 or other computer communication link to an analysis server 970.

The analysis server 970 can comprise one or more processors 974 coupled to a memory 976 which can store and retrieve instructions, and can also include a display 972. The analysis server 970 can receive the mental state data and analyze the mental state data to produce well-being status information, so that the analyzing of the mental state data can be performed by a web service. The analysis server 970 can use the mental state information received from the client machine 920 to produce a well-being status indicator. In some embodiments, the analysis server 970 receives mental state data and/or mental state information from a plurality of client machines, and aggregates the mental state information for use in optimizing the well-being status of an individual or plurality of individuals.

In some embodiments, the rendering of well-being status can occur on a different computer than the client machine 920 or the analysis server 970. In the diagram 900 this computer is labeled as a rendering machine 950, and can receive mental state data 940 including well-being status indicators from the analysis machine 970, the client machine 920, or both. In embodiments, the rendering server 950 comprises one or more processors 954 coupled to a memory 956 which can store and retrieve instructions, and a display 952. The rendering can be any visual, auditory, or other form of communication to one or more individuals. The rendering can include an email, a text message, a tone, an electrical pulse, or the like.

The system 900 can perform a computer-implemented method for mental state analysis comprising receiving mental state data on an individual, analyzing the mental state data to evaluate a well-being status for the individual, and sending the well-being status for rendering. The system 900 can further perform a computer-implemented method for physiology analysis comprising capturing mental state data on an individual, analyzing the mental state data to provide mental state information, and sending the mental state information to a server for analyzing wherein the analyzing will provide a well-being status for the individual and wherein the well-being status will be rendered. The system 900 can also perform the computer-implemented method for mental state analysis comprising receiving a well-being status based on mental state data obtained on an individual wherein the well-being status results from analyzing the mental state data to provide the well-being status for the individual, and rendering an output based on the well-being status. The system 900 can include a computer program product embodied in a non-transitory computer readable medium for mental state analysis comprising code for obtaining mental state data on an individual, code for analyzing the mental state data to evaluate a well-being status for the individual, and code for rendering an output based on the well-being status.

Each of the above methods may be executed on one or more processors on one or more computer systems. Embodiments include various forms of distributed computing, client/server computing, and cloud based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions can be stored, compiled, or interpreted to run on a computer, a programmable data processing apparatus, a heterogeneous combination of processors or processor architectures, and so on. Without limitation, embodiments of the present invention can take the form of web-based computer software, which includes client/server software, software-as-a-service, peer-to-peer software, or the like.

In embodiments, a computer can enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer can process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the forgoing examples should not limit the spirit and scope of the present invention; rather it should be understood in the broadest sense allowable by law.

What is claimed is:

1. A computer-implemented method for mental state analysis comprising:
obtaining mental state data on an individual;
analyzing the mental state data to evaluate a well-being status for the individual; and
rendering an output based on the well-being status.

2. The method of claim 1 wherein the rendering includes posting the well-being status to a social network.

3. The method of claim 2 further comprising querying for well-being statuses across the social network.

4. The method of claim 3 wherein the querying is in light of a context.

5. The method of claim 1 wherein the rendering includes providing feedback to the individual.

6. (canceled)

7. The method of claim 5 wherein the feedback describes recommended activities.

8. The method of claim 7 wherein the recommended activities include one or more of watching a video, playing a game, or participating in a social function.

9. The method of claim 5 wherein the feedback recommends eliminating an activity.

10-11. (canceled)

12. The method of claim 1 wherein the analyzing the mental state data includes evaluating frown frequency, smile frequency, or laugh frequency.

13. The method of claim 1 further comprising correlating the well-being status to activities performed by the individual.

14. The method of claim 1 further comprising calendaring the well-being status.

15. The method of claim 1 wherein the well-being status is used in emotional journaling.

16. The method of claim 1 further comprising scheduling an activity on a calendar based on the well-being status.

17-18. (canceled)

19. The method of claim 1 wherein the well-being status provides input to a recommendation engine.

20. The method of claim 1 further comprising aggregating the well-being status for the individual with well-being statuses for a plurality of other people.

21. The method of claim 20 further comprising correlating the well-being statuses with activities performed by the plurality of other people.

22. The method of claim 1 wherein the analyzing includes evaluation of an impaired state.

23. (canceled)

24. The method of claim 1 wherein the mental state data includes facial data, physiological data, or accelerometer data.

25. (canceled)

26. The method of claim 24 wherein the physiological data includes one or more of heart rate, heart rate variability, blink rate, electrodermal activity, skin temperature, and respiration.

27. The method of claim 24 wherein the physiological data is derived from a biosensor.

28. The method of claim 1 wherein the well-being status is used for advertisement selection.

29. The method of claim 1 wherein the well-being status is used to modify a game.

30. The method of claim 1 wherein the well-being status is used to modify a media presentation.

31. The method of claim 1 further comprising inferring mental states based on the mental state data which was obtained wherein the mental states include one or more of frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity.

32. (canceled)

33. The method of claim 1 wherein the mental state data is obtained from multiple sources.

34. The method of claim 33 wherein at least one of the multiple sources is a mobile device.

35. The method of claim 1 wherein the mental state data is collected sporadically.

36. The method of claim 1 wherein the analyzing of the mental state data is performed by a web service.

37. The method of claim 1 further comprising determining context during which the mental state data is captured.

38. A computer program product embodied in a non-transitory computer readable medium for mental state analysis, the computer program product comprising:
code for obtaining mental state data on an individual;
code for analyzing the mental state data to evaluate a well-being status for the individual; and
code for rendering an output based on the well-being status.

39. A computer system for mental state analysis comprising:
a memory which stores instructions;
one or more processors attached to the memory wherein the one or more processors, when executing the instructions which are stored, are configured to:
obtain mental state data on an individual;
analyze the mental state data to evaluate a well-being status for the individual; and
render an output based on the well-being status.

40-42. (canceled)
